(19) United States
(12) Patent Application Publication
(10) Pub. No.: US 2006/0057730 A1
(43) Pub. Date: Mar. 16, 2006

(54) Method for protein determination, indicator for protein determination, and test piece for protein determination

(75) Inventor: Hideko Kosaka, Kyoto (JP)

Correspondence Address: MERCHANT & GOULD PC, P.O. BOX 2903, MINNEAPOLIS, MN, 55402-0903, US

(73) Assignee: ARKRAY, INC., Kyoto (JP)

(21) Appl. No.: 10/523,865

(22) PCT Filed: Aug. 4, 2003
(86) PCT No.: PCT/JP03/09888

§ 371 Date: Feb. 7, 2005

(30) Foreign Application Priority Data
Aug. 9, 2002 (JP) 2002-233467

(51) Int. Cl. G01N 33/48

(52) U.S. Cl. 436/86; 436/166

(57) ABSTRACT

The present invention relates to a technique for assaying a protein. With the present invention, a compound having a chemical structure expressed by the following Chemical Formula (1) is used as a protein assay indicator. In Chemical Formula (1), X1 is a halogen, a nitro group, or a nitroso group; X2 is a halogen; X3 is a halogen or hydrogen; X4 is a hydroxyl group or a salt thereof, and X5 is a carboxyl group or a salt thereof.

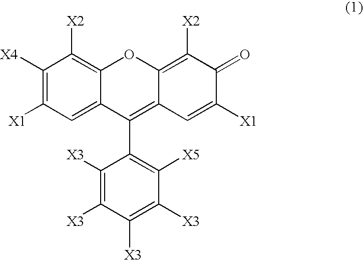

### TECHNICAL FIELD

This invention relates to a technique for assaying proteins present in protein-containing samples (body fluid such as blood and urine, or protein-containing beverages, or factory wastewater, etc.).

### BACKGROUND ART

Assaying the protein in a biological sample is important in pathological diagnosis. For instance, the amount of serum albumin decreases in the case of diminished liver function, while the amount of protein in urine increases in the case of nephritis, nephrotic syndrome, lithiasis, tumors, and other kidney and urinary tract disorders, disorders of the circulatory system and disorders of central nervous system. Therefore, assaying albumin or other proteins can be an important clue in the diagnosis of these disorders.

A simple assay method featuring the use of a protein error indicator is known in the field of protein assay. With this assay method, tetrabromophenol blue (TBPB) is used, for instance, as the protein error indicator. As one example, urine test paper made with TBPB is widely used for primary screening purposes. TBPB changes from yellow to blue through the dissociation of phenolic hydroxyl groups at a pH of about 3 when a protein is present, and therefore can be used to detect protein.

However, test paper made using TBPB as the indicator has inadequate sensitivity with respect to the low protein concentrations of 10 to 20 mg/dL required for clinical use, and is therefore sometimes incapable of detecting protein accurately. For example, in a visual evaluation conducted by comparison with a color chart, the color is very similar between negative protein and trace protein, making it difficult to tell the two apart and hampering accurate evaluation. Meanwhile, when a urine test paper assay apparatus is used, the low sensitivity of TBPB often results in erroneous evaluation.

Consequently, there has been a need for a technique that would allow low concentrations of protein to be quantified at higher sensitivity, and more particularly for the development of a novel indicator other than TBPB.

### DISCLOSURE OF THE INVENTION

As a result of screening indicators for assaying low concentrations of protein at high sensitivity, the inventors arrived at the present invention upon discovering that a specific halogenated xanthene-based dye is favorable as the targeted indicator.

Specifically, the present invention provides a protein assay indicator having the chemical structure expressed by the following Chemical Formula (1), and a protein assay method and test piece for protein assay that make use of this protein assay indicator.

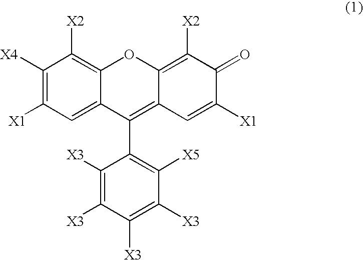

In Chemical Formula (1), X1 is a halogen, a nitro group, or a nitroso group; X2 is a halogen; X3 is a halogen or hydrogen; X4 is a hydroxyl group or a salt thereof; and X5 is a carboxyl group or a salt thereof. With the present invention, it is preferable for the protein assay indicator to be such that, in Chemical Formula (1), X1 is iodine, bromine, chlorine, or a nitro group, X2 is iodine or bromine, and X3 is chlorine, bromine, or hydrogen. Ideally, X1 and X2 are each iodine or bromine, and X3 is chlorine. A typical example of the salts in X4 and X5 is a sodium salt.

Typical examples of the protein assay indicator of the present invention include those expressed by the following Chemical Formulas (1)-1 to (1)-5. Of these, the protein assay indicators of the following Chemical Formulas (1)-1 and (1)-2 are preferable.

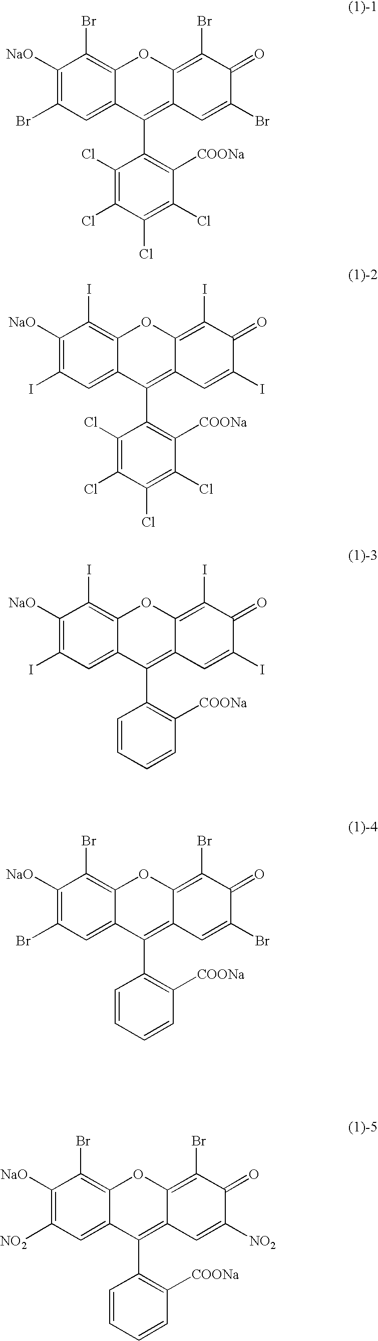

These halogenated xanthene-based dyes are from colorless to light orange in color when no protein is present at a pH at or below the pKa of said dye, but are from red to purple in color when a protein is present. Accordingly, since the original coloring varies from colorless to a pale color, a change in color is easier to detect than when the color changes from yellow to blue as with TBPB. Therefore, if one of the above-mentioned halogenated xanthene-based dyes is used, low-concentration proteins can be detected properly regardless of whether the evaluation is visual or a measurement apparatus is used.

The test piece for protein assay of the present invention can be manufactured by impregnating an absorbent carrier with an impregnant which contains the above-mentioned halogenated xanthene-based dye, a buffer, a sensitizer, or the like, and then drying this product. This test piece can be used directly as it is, or after first being bonded to a non-absorbent material.

There are no particular restrictions on the concentration of the halogenated xanthene-based dye in the impregnant, but it is typically 0.1 to 10 mM, and preferably 0.5 to 2 mM.

The pH of the impregnant is set between 1.5 and 4.5, which is somewhat lower than the pKa of the halogenated xanthene-based dye of the present invention, and is preferably from 2.0 to 3.5.

Any buffer can be used as long as it has a good buffering action within a pH range of 1.5 to 4.5 and does not impede reaction between the protein and the halogenated xanthene-based dye. Examples of buffers that can be used include glycine buffer, citrate buffer, succinate buffer, malate buffer, and tartrate buffer. There are no particular restrictions on the concentration of the buffer in the impregnant, but it is typically from 0.1 to 1.5 M, and preferably 0.3 to 1 M.

Examples of sensitizers that can be used include polyethylene glycol, polypropylene glycol, polycarbonate, and polyvinyl ether. The use of polyethylene glycol or polypropylene glycol is preferred. There are no particular restrictions on the concentration of the sensitizer in the impregnant, but it is typically from 0.05 to 5 wt %, and preferably 0.1 to 1 wt %.

A porous substance containing no protein component can be used as the absorbent carrier, and can be used in the form of a sheet or film, for example. Examples of porous substances include paper-like materials, foams, woven materials, nonwoven materials, and knits. Examples of the material used to form the absorbent carrier include cotton, linen, cellulose, nitrocellulose, cellulose acetate, rock wool, glass fiber, silica fiber, carbon fiber, boron fiber, polyamide, aramid, polyvinyl alcohol, polyvinyl acetate, rayon, polyester, nylon, polyacrylic acid, polyacrylic ester, and polyolefin. There are no particular restrictions on the shape of the absorbent carrier, but it is generally rectangular (either short and wide or long and narrow), circular, or oval.

The non-absorbent material is used in the form of a sheet or film, for example. Examples of the material used to form this non-absorbent material include polyethylene terephthalate, polyester, polypropylene, polyethylene, polyvinyl chloride, polyvinylidene chloride, and polystyrene.

### EXAMPLES

### Example 1

In this example, indicators were screened for their ability to detect low concentrations of protein. This screening was performed by adding the test compound such that its concentration in the screening solution would be 0.5 mM, and visually observing the resulting coloration. The screening solution was prepared by dissolving 15 mg/dL albumin and 0.5 wt % polyethylene glycol in a 0.7 M malate buffer (pH 2.2). The test compounds were various commercially available dyes. As a result, good coloration was seen with the five compounds expressed by the following Chemical Formulas (2)-1 to (2)-5. Table 1 shows where these compounds were obtained.

TABLE 1

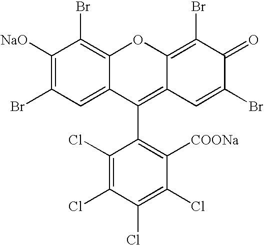

(2)-1

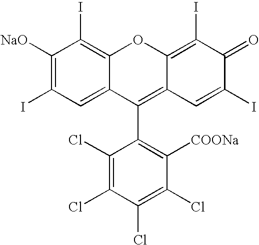

(2)-2

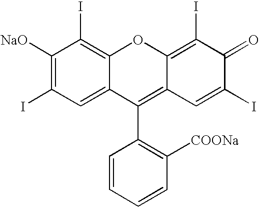

(2)-3

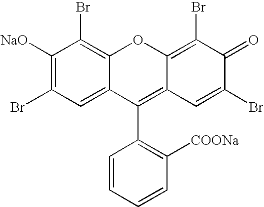

(2)-4

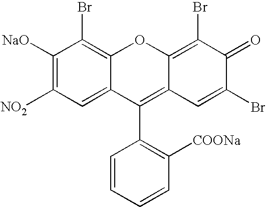

(2)-5

| Chemical Formula No. | Product name | Manufacturer |
|---|---|---|
| (2)-1 | phloxine B | Tokyo Chemical Industries |
| (2)-2 | rose bengal | Tokyo Chemical Industries |
| (2)-3 | erythrosine B | Tokyo Chemical Industries |
| (2)-4 | eosin Y | Wako Pure Chemical Industries |
| (2)-5 | eosin B | Tokyo Chemical Industries |

### Example 2

In this example, the sensitivity of the screened indicators was evaluated. This was accomplished by impregnating each test piece with urine whose albumin concentration was either 0.3 mg/dL (negative) or 15 mg/dL (positive), and measuring the reflectance of each. The test pieces were formed by impregnating filter paper (3 MMChr made by Whatman) with an impregnant having the composition given in Table 2. Reflectance was measured with a colorimeter. Table 3 shows the measurement results for the various samples. Table 3 also shows the measurement wavelength for each sample.

TABLE 2

| | Indicator | Buffer | Sensitizer | Solvent |
|---|---|---|---|---|
| Sample 1 | phloxine B | malate buffer | polyethylene | ethanol |
| | (0.5 mM) | 0.7 M (pH 2.2) | glycol 0.5 wt % | (40 wt %) |
| Sample 2 | phloxine B | malate buffer | none | ethanol |
| | (0.5 mM) | 0.7 M (pH 2.2) | | (40 wt %) |
| Sample 3 | rose bengal | malate buffer | polyethylene | ethanol |
| | (0.5 mM) | 0.7 M (pH 2.6) | glycol 0.5 wt % | (40 wt %) |
| Sample 4 | rose bengal | malate buffer | none | ethanol |
| | (0.5 mM) | 0.7 M (pH 2.6) | | (40 wt %) |
| Sample 5 | TBPB | malate buffer | polyethylene | ethanol |
| | (0.5 mM) | 0.7 M (pH 3.4) | glycol 0.5 wt % | (30 wt %) |
| Sample 6 | TBPB | malate buffer | none | ethanol |
| | (0.5 mM) | 0.7 M (pH 3.4) | | (30 wt %) |

Note:
TBPB = tetrabromophenol blue

TABLE 3

| | | Reflectance (%) | | |
|---|---|---|---|---|
| | Measurement | 0.3 mg/dL | 15 mg/dL | |
| | wavelength | (negative) | (positive) | Differential Δ |
| Sample 1 | 560 nm | 65.8 | 38.2 | 27.7 |
| Sample 2 | 560 nm | 61.5 | 38.3 | 23.2 |
| Sample 3 | 560 nm | 65.7 | 40.7 | 25.0 |
| Sample 4 | 560 nm | 61.6 | 41.6 | 20.0 |
| Sample 5 | 630 nm | 57.0 | 40.4 | 16.6 |
| Sample 6 | 630 nm | 60.7 | 48.9 | 11.8 |

As is clear from Table 3, with samples 1 and 2 in which the phloxine B expressed by Chemical Formula (2)-1 was used as the indicator, and samples 3 and 4 in which the rose bengal expressed by Chemical Formula (2)-2 was used, the reflectance was higher when the albumin concentration was 0.3 mg/dL (negative) and lower when the albumin concentration was 15 mg/dL (positive) than with samples 5 and 6, in which TBPB was used as the indicator. In other words, phloxine B and rose bengal absorbed less light during non-coloration (negative), and conversely absorbed more light during coloration (positive), than TBPB because they were colorless under these pH conditions. Accordingly, phloxine B and rose bengal have a large reflectance differential A between negative urine and positive urine, with this differential being about twice that with TBPB. Therefore, phloxine B and rose bengal can be said to have higher sensitivity with respect to albumin, and allow albumin to be properly detected even when the albumin concentration is low (about 10 to 20 mg/dL).

The inventors also checked whether the samples could be evaluated visually. As a result, samples 1 to 4, in which phloxine B and rose bengal were used, changed from colorless to red when the albumin concentration was 15 mg/dL, and this coloration (positive) could be easily confirmed. In contrast, with samples 5 and 6, in which TBPB was used, there was almost no difference from the negative yellow color when the albumin concentration was 15 mg/dL, making it very difficult to visually discern any coloration. Thus, when phloxine B and rose bengal were used as the indicator, albumin could be easily detected visually, even at a low albumin concentration.

The experiment results in Example 2 pertain to urine samples, but the present invention is not limited to urine, and can also be applied to the quantification of protein in any of various other samples containing protein, such as blood, protein-containing beverages, and factory wastewater.

1. A method for assaying a protein by using a protein assay indicator,
wherein a compound having a chemical structure expressed by the following Chemical Formula (1) is used as the protein assay indicator: 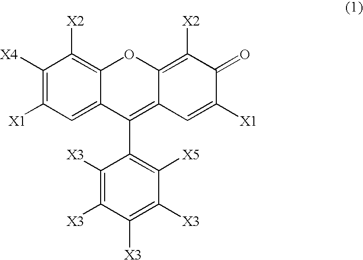
where, in Chemical Formula (1), X1 is a halogen, a nitro group, or a nitroso group; X2 is a halogen; X3 is a halogen or hydrogen; X4 is a hydroxyl group or a salt thereof; and X5 is a carboxyl group or a salt thereof.

2. The protein assay method according to claim 1, wherein, in Chemical Formula (1), X1 is iodine, bromine, chlorine, or a nitro group, X2 is iodine or bromine, and X3 is chlorine, bromine, or hydrogen.

3. The protein assay method according to claim 2, wherein, in Chemical Formula (1), X1 and X2 are iodine or bromine, and X3 is chlorine.

4. The protein assay method according to claim 3, wherein at least one compound selected from the group of compounds expressed by the following Chemical Formulas (1)-1 to (1)-5 is used as the protein indicator.

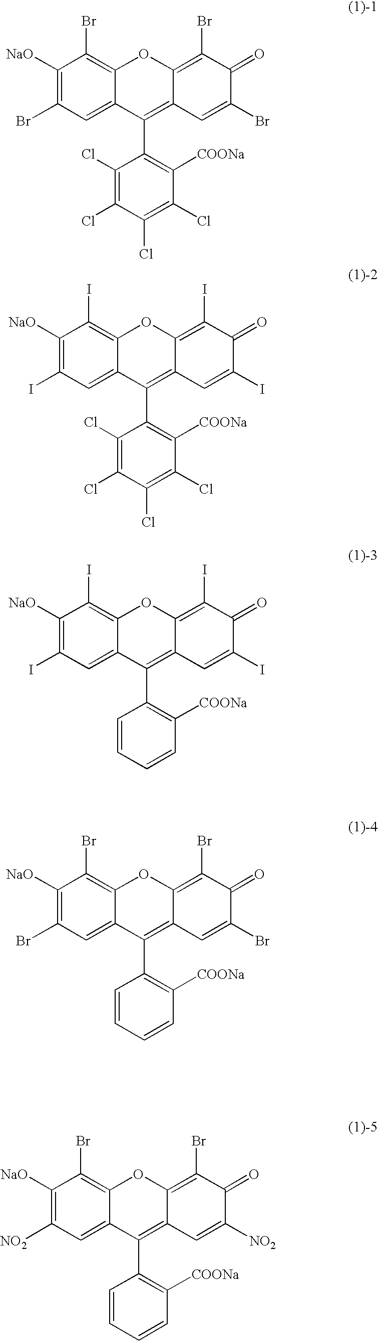

5. The protein assay method according to claim 1, wherein the protein indicator is from colorless to light orange in color when no protein is present at a pH equal to or below the pKa of said protein indicator, but is from red to purple in color when a protein is present.

6. The protein assay method according to claim 1, wherein the protein is albumin.

7. The protein assay method according to claim 6, wherein albumin concentration is measured for an albumin-containing sample whose albumin concentration is between 10 and 20 mg/dL.

8. A protein assay indicator for assaying a protein, said indicator having a chemical structure expressed by the following Chemical Formula (2): 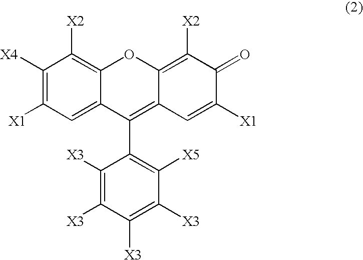
where, in Chemical Formula (2), X1 is a halogen, a nitro group, or a nitroso group; X2 is a halogen; X3 is a halogen or hydrogen; X4 is a hydroxyl group or a salt thereof; and X5 is a carboxyl group or a salt thereof.

9. The protein assay indicator according to claim 8, wherein, in Chemical Formula (2), X1 is iodine, bromine, chlorine, or a nitro group, X2 is iodine or bromine, and X3 is chlorine, bromine, or hydrogen.

10. The protein assay indicator according to claim 9, wherein, in Chemical Formula (2), X1 and X2 are iodine or bromine, and X3 is chlorine.

11. The protein assay indicator according to claim 8, the indicator being at least one compound selected from the group of compounds expressed by the following Chemical Formulas (2)-1 to (2)-5:

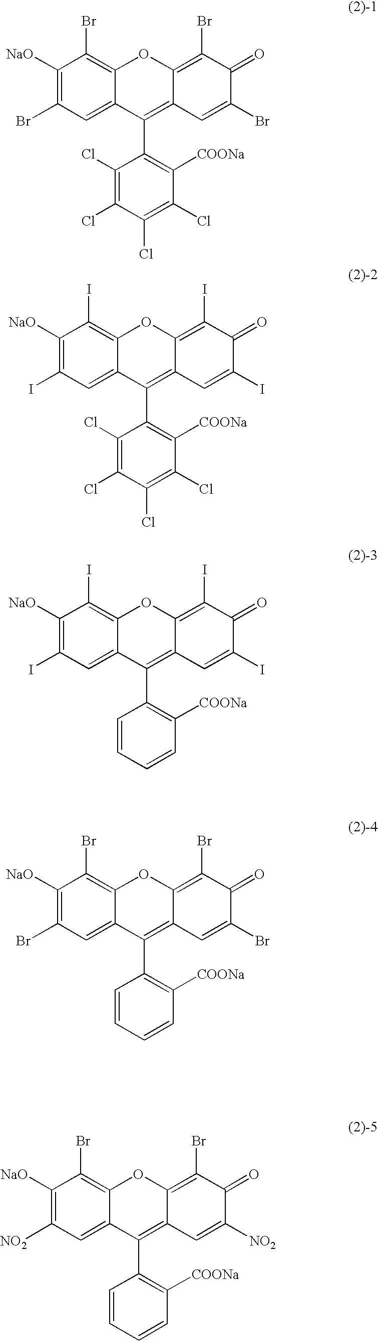

12. The protein assay indicator according to claim 8, wherein the indicator being is from colorless to light orange in color when no protein is present at a pH equal to or below the pKa, but is from red to purple in color when a protein is present.

13. A test piece for protein assay used for quantifying or semi-quantifying a protein, wherein a compound having a chemical structure expressed by the following Chemical Formula (3) is used as a protein assay indicator: 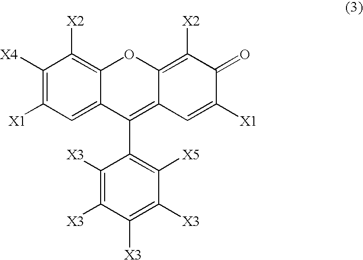
where, in Chemical Formula (3), X1 is a halogen, a nitro group, or a nitroso group; X2 is a halogen; X3 is a halogen or hydrogen; X4 is a hydroxyl group or a salt thereof; and X5 is a carboxyl group or a salt thereof.

14. The test piece for protein assay according to claim 13, wherein, in Chemical Formula (3), X1 is iodine, bromine, chlorine, or a nitro group, X2 is iodine or bromine, and X3 is chlorine, bromine, or hydrogen.

15. The test piece for protein assay according to claim 13, wherein, in Chemical Formula (3), X1 and X2 are iodine or bromine, and X3 is chlorine.

16. The test piece for protein assay according to claim 13, wherein at least one compound selected from the group of compounds expressed by the following Chemical Formulas (3)-1 to (3)-5 is used as the protein indicator:

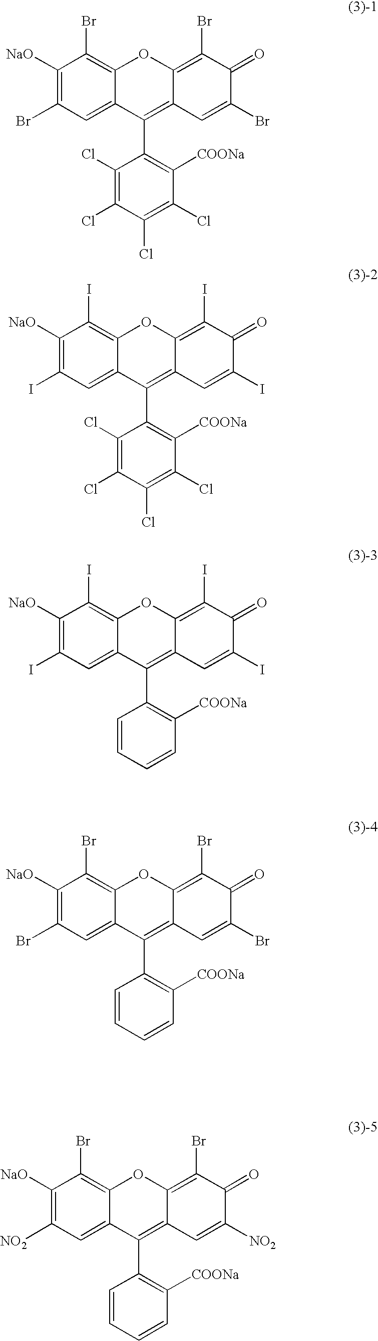

17. The test piece for protein assay according to claim 13, wherein the protein indicator is from colorless to light orange in color when no protein is present at a pH equal to or below the pKa of said protein indicator, but is from red to purple in color when a protein is present.

18. The test piece for protein assay according to claim 13, further containing a sensitizer for increasing the coloration sensitivity with respect to the protein.

19. The test piece for protein assay according to claim 18, containing polyethylene glycol and/or polypropylene glycol as the sensitizer.
